(19) United States
(12) Patent Application Publication
(10) Pub. No.: US 2018/0225634 A1
(43) Pub. Date: Aug. 9, 2018

(54) DATA ANALYSIS DEVICE, DATA ANALYSIS METHOD, AND STORAGE MEDIUM STORING DATA ANALYSIS PROGRAM

(71) Applicants: NEC Corporation, Minato-ku, Tokyo (JP); NEC SOLUTION INNOVATORS, LTD., Koto-ku, Tokyo (JP)

(72) Inventors: Yuki KOSAKA, Tokyo (JP); Hiroaki FUKUNISHI, Tokyo (JP); Hirofumi TANAKA, Tokyo (JP); Masashi NAKAMICHI, Tokyo (JP); Haruka OKAMOTO, Tokyo (JP)

(73) Assignees: NEC Corporation, Minato-ku, Tokyo (JP); NEC SOLUTION INNOVATORS, LTD., Koto-ku, Tokyo (JP)

(21) Appl. No.: 15/742,948

(22) PCT Filed: Jul. 14, 2016
(86) PCT No.: PCT/JP2016/003332

§ 371 Date: Jan. 9, 2018

(30) Foreign Application Priority Data
Jul. 16, 2015 (JP) 2015-142404

(51) Int. Cl. G06Q 10/10; G16H 50/30; G06Q 10/06; G06F 15/18

(52) U.S. Cl. CPC G06Q 10/1091; G06F 15/18; G06Q 10/063; G16H 50/30

(57) ABSTRACT

A data analysis device includes: a data acquiring unit that acquires a designation of a target field being a field from which relevance is to be extracted, from among fields included in health condition data being information relating to a health condition of an employee, and the health condition data of two or more employees and attendance data being information relating to a work condition; an attribute data generating unit that performs aggregation, and generates attribute data; a model learning unit that learns a model, the model being represented by a polynomial, by using a content of the target field of the health condition data, and a content of the attribute data, of the two or more employees; a related field extracting unit that extracts an attribute field; and a summarizing unit that summarizes and outputs attendance data, based on information on the extracted attribute field.

### TECHNICAL FIELD

The disclosed subject matter relates to a data analysis device, a data analysis method, and a storage medium storing a data analysis program for supporting health guidance of a company and the like.

### BACKGROUND ART

Maintaining or promoting health of employee or person who belongs to an organization for carrying out business (hereinafter, simply referred to as “employee”) is one of very important roles to an employer or a person who manages the organization (hereinafter, simply referred to as an “employer”). In view of the above, the employer provides a health care worker such as an industrial physician and a public health nurse, and implements many measures relating to medical examination or health guidance for an employee.

The health care worker provides an advice for health promotion to an employee as health guidance, based on a medical examination result or an interview result of the employee relating to a lifestyle habit such as a diet, an exercise habit, a sleep habit, and a smoking habit.

A device has been developed for the purpose of efficiency of health guidance performed for the employee by the health care worker. The device extracts features on health and the lifestyle habit of the employee from a medical examination result of the employee or an interview result relating to the lifestyle habit.

For example, PTL 1 describes a health support system, in which a plurality of individuals who ask for an advice are grouped based on medical examination results and interview results on lifestyle habits, of the individuals, and an advice for health maintenance/promotion is provided, based on features on health conditions and lifestyle habits extracted for each group.

For example, using the technology described in PTL 1 makes it possible to provide an advice such that an individual belonging to a group in which a blood pressure is high as compared with other groups needs to take less salty meals in order to lower the blood pressure, from a medical point of view.

### CITATION LIST

### Patent Literature

[PTL 1] Japanese Laid-open Patent Publication No. 2010-170534

### SUMMARY OF INVENTION

### Technical Problem

A health condition of the employee depends on a lifestyle habit in many cases. Therefore, it is important to grasp a factor of deterioration of the lifestyle habit in order to perform effective health guidance.

As the factor of deterioration of the lifestyle habit, deterioration of basic matters (mainly, matters relating to a living condition) in daily life of the employee, such as a diet, exercise, and sleep is exemplified. However, overwork such as long working hours or irregular work shifts at an office may be related to deterioration of the lifestyle habit. For example, a body of the employee may suffer from a serious disease unconsciously, triggered by mental stress at work or in an office environment.

In view of the above, in order to effectively provide the advice to the employee, it is important to accurately grasp/comprehend a work situation such as daily overtime hours, frequency of taking a day off, and frequency of holiday work, in addition to the medical examination result or the interview result relating to the lifestyle habit such as the diet, exercise, and sleep, of the employee.

It is often the case that an advisor such as a health care worker who performs health guidance utilizes attendance data as an important information source for checking a work situation of an employee. The attendance data is information, in which matters relating to a work situation of each employee, are arranged in a time series manner such as daily arrival times, daily leaving times, presence or absence of work, presence or absence of a day off, and overtime hours.

Generally, attendance data includes several tens of fields. Further, in many of the fields, data are recorded and increase in such a manner that one record is added per day. The number of records of attendance data tends to increase, in addition to the number of fields, but time of health guidance for each employee is limited. Therefore, it is difficult for an advisor to check all these pieces of information within a limited time.

As described above, there is a problem that an advisor may not easily obtain, from attendance data, a concrete work situation associated with a health condition (e.g. presence or absence of overwork, irregular work shifts, or the like, or a degree thereof), since an amount of attendance data to check is large.

PTL 1 describes generating a plurality of health condition groups by covariance structure analysis from data relating to health conditions and management thereof, and presenting feature characteristics to persons belonging to these health condition groups, as recommended item data for use in staying in the group or moving to another group. According to the aforementioned configuration, a health director is able to provide an advice such as presenting recommended behavior information, based on presented recommended item data.

However, the method described in PTL 1 only extracts an item indicating a feature characteristic belonging to each group, and fails to extract an item appropriate for an intention of an advisor, a degree of relevance of the item, and the like. For example, it is assumed that an advisor focuses on a certain symptom, and wishes that fields other than a field particularly associated with the symptom are not presented among attendance data. In this case, there is no guarantee that grouping is performed depending on presence or absence of the symptom or a degree of the symptom, even when the method described in PTL 1 is applied. Further, an advisor may focus on another symptom at another timing, and may wish that fields other than a field particularly associated with the symptom are not presented among attendance data. However, PTL 1 does not describe a method for appropriately summarizing (such as selecting and processing information) and presenting attendance data in accordance with an intention of an advisor at all.

In view of the above, an object of the disclosed subject matter is to provide a data analysis device, a data analysis method, and a data analysis program which allow an advisor to easily obtain concrete field information included in data associated with a health condition of an employee including attendance data, which are associated with any item focused by the advisor.

### Solution to Problem

According to one aspect of the disclosed subject matter, a data analysis device includes: data acquiring means for acquiring at least designation of a target field being a field from which relevance is to be extracted, from among fields included in health condition data being information relating to a health condition of an employee, and the health condition data of two or more employees and attendance data being information relating to a work condition; attribute data generating means for performing aggregation with respect to a predetermined field included in the attendance data of each of the employees by using a predetermined temporal resolution, a time range, and an aggregation method, and generating attribute data including each of aggregation results as an attribute field; model learning means for learning a model, in which the target field is an object variable, and each of attribute fields included in the attribute data is an explanatory variable, the model being represented by a polynomial, by using a content of the target field of the health condition data, and a content of the attribute data, of the two or more employees; related field extracting means for extracting an attribute field represented by a learned model and associated with the target field; and summarizing means for summarizing and outputting attendance data of a designated employee, based on information on the extracted attribute field.

According to one aspect of the disclosed subject matter, A data analysis method includes: causing an information processing device to acquire at least designation of a target field being a field from which relevance is to be extracted, from among fields included in health condition data being information relating to a health condition of an employee, and the health condition data of two or more employees and attendance data being information relating to a work condition; causing the information processing device to perform aggregation with respect to a predetermined field included in the attendance data of each of the employees by using a predetermined temporal resolution, a time range, and an aggregation method, and to generate attribute data including each of aggregation results as an attribute field; causing the information processing device to learn a model, in which the target field is an object variable, and each of attribute fields included in the attribute data is an explanatory variable, the model being represented by a polynomial, by using a content of the target field of the health condition data, and a content of the attribute data, of the two or more employees; causing the information processing device to extract an attribute field represented by a learned model and associated with the target field; and causing the information processing device to summarize and output attendance data of a designated employee, based on information on the extracted attribute field.

According to one aspect of the disclosed subject matter, a storage medium storing a data analysis program which causes a computer to execute: processing of acquiring at least designation of a target field being a field from which relevance is to be extracted, from among fields included in health condition data being information relating to a health condition of an employee, and the health condition data of two or more employees and attendance data being information relating to a work condition; processing of performing aggregation with respect to a predetermined field included in the attendance data of each of the employees by using a predetermined temporal resolution, a time range, and an aggregation method, and generating attribute data including each of aggregation results as an attribute field; processing of learning a model, in which the target field is an object variable, and each of attribute fields included in the attribute data is an explanatory variable, the model being represented by a polynomial, by using a content of the target field of the health condition data, and a content of the attribute data, of the two or more employees; processing of extracting an attribute field represented by a learned model and associated with the target field; and processing of summarizing and outputting attendance data of a designated employee, based on information on the extracted attribute field.

### Advantageous Effects of Invention

According to the disclosed subject matter, an advisor is able to easily obtain concrete field information included in data associated with a health condition of an employee including attendance data, which are associated with any item focused by the advisor.

### BRIEF DESCRIPTION OF DRAWINGS

FIG. 1 is a block diagram illustrating a configuration example of a data analysis device in a first example embodiment;

FIG. 2 is a configuration diagram illustrating an example of a hardware configuration of a data analysis device 10;

FIG. 3 is a flowchart illustrating an example of an operation of the data analysis device 10 in the first example embodiment;

FIG. 4 is an explanatory diagram illustrating a time series relationship between attendance data, and a temporal resolution with respect to the attendance data;

FIG. 5 is an explanatory diagram illustrating an example of attendance data;

FIG. 6 is an explanatory diagram illustrating an example of attribute data setting information;

FIG. 7 is an explanatory diagram illustrating an example of attribute data;

FIG. 8 is an explanatory diagram illustrating an example of an attribute table including model parameters obtained as a result of learning;

FIG. 9 is an explanatory diagram illustrating an example of a summary result of attendance data;

FIG. 10 is an explanatory diagram illustrating an example of attendance data in a first modification example;

FIG. 11 is an explanatory diagram illustrating another example of attendance data in the first modification example;

FIG. 12 is a block diagram illustrating a configuration example of a data analysis device in a second modification example;

FIG. 13 is a flowchart illustrating an example of an operation of the data analysis device in the second modification example;

FIG. 14 is an explanatory diagram illustrating an example of attribute data setting information in a third modification example;

FIG. 15 is an explanatory diagram illustrating an example of attribute data in the third modification example;

FIG. 16 is an explanatory diagram illustrating another example of an attribute table;

FIG. 17 is an explanatory diagram illustrating an example of a summary result of attendance data and medical examination data;

FIG. 18 is an explanatory diagram illustrating a relationship between attendance data and a medical examination day in the third modification example;

FIG. 19 is a block diagram illustrating a configuration example of a data analysis device in a fourth modification example;

FIG. 20 is an explanatory diagram illustrating an example of grouping in the fourth modification example; and

FIG. 21 is a block diagram illustrating a summary of a data analysis device according to the disclosed subject matter.

### DESCRIPTION OF EMBODIMENTS

### First Example Embodiment

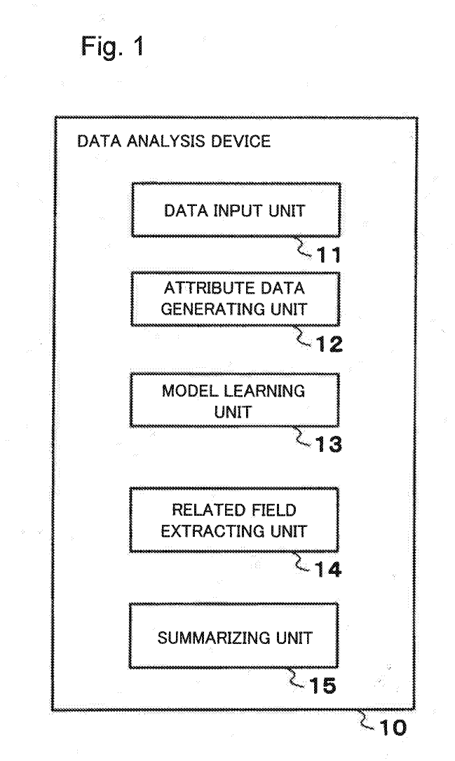

In the following, an example embodiment of the disclosed subject matter is described with reference to the drawings. FIG. 1 is a block diagram illustrating a configuration example of a data analysis device in the first example embodiment of the disclosed subject matter.

The data analysis device 10 illustrated in FIG. 1 includes a data input unit 11, an attribute data generating unit 12, a model learning unit 13, a related field extracting unit 14, and a summarizing unit 15.

The data input unit 11 receives information necessary for each processing unit of the data analysis device 10.

Input information may include, for example, designation of a target field, health condition data in the past and attendance data, or the like. The designation of the target field is a field from which relevance is extracted, from among fields included in health condition data being data relating to health conditions of an employee. The attendance data includes data for a predetermined period earlier than a data measurement day being a day when the health condition data are measured.

In the following, there is described, by using an example, a case where health condition data are medical examination data indicating a result of medical examination (inspection), or an interview relating to health of an employee, and a data measurement day is a medical examination day (when there are a plurality of days, one of these days) that is a day when the medical examination data are acquired. The health condition data and the data measurement day are not limited to these. Further, the attendance data are also included in health condition data in a broad sense.

For example, the data input unit 11 may receive information indicating an attribute data generation method as will be described later, in addition to the aforementioned information.

The target field may be any of fields included in the health condition data, and may also be a plurality of fields.

The attribute data generating unit 12 generates attribute data indicating various features on a work condition of each employee, based on attendance data received. More specifically, the attribute data generating unit 12 generates attribute data obtained by aggregating information in various time ranges of each employee for each field of attendance data at a predetermined temporal resolution such as every month, every quarter of a year, every half a year, or every year. An aggregation method, specifically, a calculation method for use in aggregation is not limited to one, and a plurality of calculation methods may be used. Further, it is preferable to perform aggregation by using a plurality of temporal resolutions and time ranges with respect to one attendance field.

The model learning unit 13 learns a model represented by a polynomial for use in calculating a value of an object variable from a value of each explanatory variable, by using medical examination data received and attendance data received of a plurality of employees. The object variable a target field, and the explanatory variable is each field of attribute data (hereinafter, referred to as an attribute field). The model learning unit 13 specifically learns a coefficient of each explanatory variable in the polynomial.

The related field extracting unit 14 extracts an attribute field represented by a learned model and associated with a target field. The related field extracting unit 14 may specifically extract an attribute field associated with an explanatory variable having a coefficient value other than zero. The related field extracting unit 14 may extract, as extracted attribute field information, information relating to an attendance field used in generating the attribute field, and a summarizing method with respect to the field (such as a temporal resolution, a time range, and an aggregation method with respect to the attendance field). In addition to the above, the related field extracting unit 14 may extract a coefficient value, as information indicating a degree of relevance.

The summarizing unit 15 summaries and outputs attendance data, based on an extraction result by the related field extracting unit 14. For example, the summarizing unit 15 may output by excluding a field other than an attendance field used in generating an extracted attribute field from attendance data of a designated employee. For example, the summarizing unit 15 may output an aggregation result with respect to an attendance field used in generating an extracted attribute field, from among attendance data of a designated employee, by using an extracted temporal resolution, an extracted time range, and an extracted aggregation method together with a coefficient value.

Note that when a temporal resolution is one day, or the like, it is assumed that a case where original data are output as it is included in “aggregation”. Further, an aggregation result may already be held as an attribute value of an attribute field. In this case, the summarizing unit 15 may omit aggregation processing.

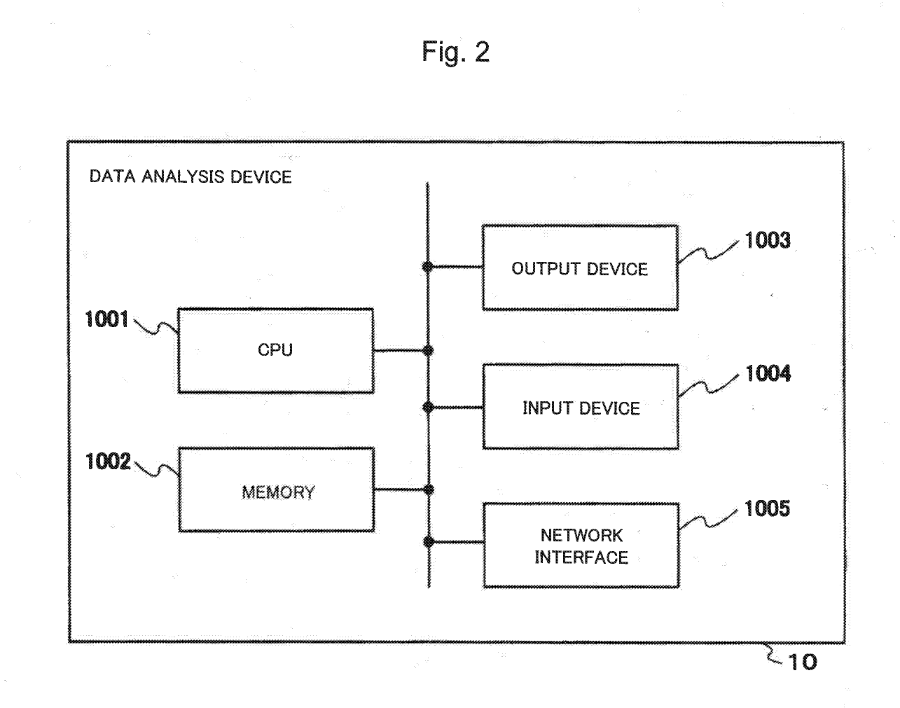

Further, FIG. 2 is a configuration diagram illustrating an example of a hardware configuration of the data analysis device 10. The data analysis device 10 illustrated in FIG. 2 includes a Central Processing Unit (CPU) 1001, a memory 1002, an output device 1003, an input device 1004, and a network interface 1005.

The memory 1002 is, for example, a Random Access Memory (RAM), a Read Only Memory (ROM), an auxiliary storage device (such as a hard disk), or the like. The output device 1003 is, for example, a device for outputting information such as a display device or a printer. The input device 1004 is, for example, a device for receiving an input of a user operation such as a keyboard or a mouse. The network interface 1005 is, for example, an interface to be connected to a network configured by the Internet, a Local Area Network (LAN), a public network, a wireless communication network, combination of these networks, or the like.

For example, each of the aforementioned functional blocks of the data analysis device 10 illustrated in FIG. 1 is configured by the CPU 1001 which executes a computer program stored in the memory 1002 by reading, and which controls each of the other units. Note that a hardware configuration of the data analysis device 10 and each functional block thereof is not limited to the aforementioned configuration.

Note that the data input unit 11 may read the aforementioned input information from the memory 1002, in addition to receive from an outside.

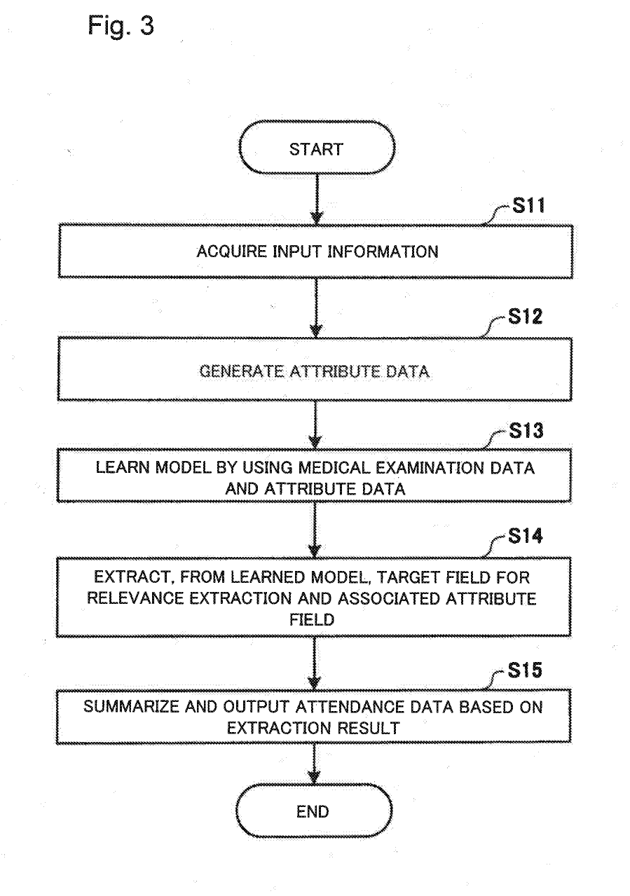

Next, an operation of the present example embodiment is described. FIG. 3 is a flowchart illustrating an example of an operation of the data analysis device 10 of the present example embodiment. In the example illustrated in FIG. 3, first of all, a data input unit 11 designates a target field, and receives medical examination data and attendance data of each employee (Step S11).

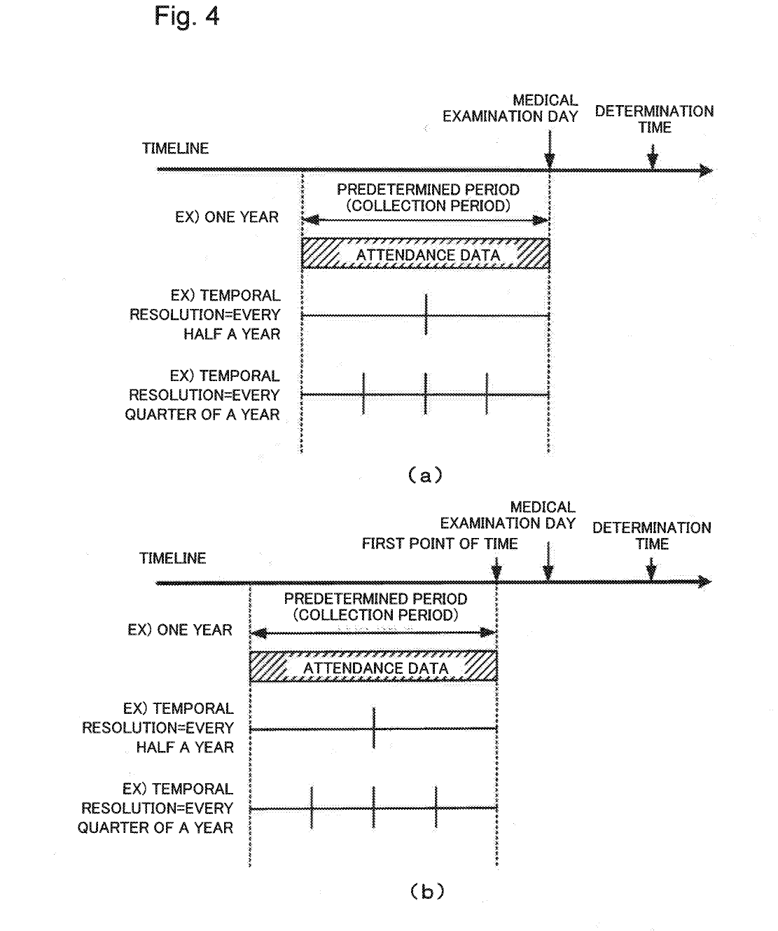

FIG. 4 is an explanatory diagram illustrating a time series relationship between attendance data as input information, and a temporal resolution with respect to the attendance data. As illustrated in FIG. 4(a), attendance data may include records for a predetermined period (e.g. for one year) earlier than a latest medical examination day before a determination time (e.g. a point of time when an advice is provided). Note that FIG. 4(a) illustrates an example, in which a last day of the aforementioned predetermined period is a medical examination day. Any number of days may be provided between a predetermined period serving as an attendance data collection period, and a medical examination day. For example, as illustrated in FIG. 4(b), attendance data may include records for a predetermined period before a first point of time by assuming that any point of time (e.g. end of year) earlier than a latest medical examination day is the first point of time. Further, a medical examination day is not limited to a latest day. In other words, attendance data may include records in a time range including a predetermined period, as far as the time range does not exceed a day (medical examination day) when a content of a target field from which relevance is extracted is acquired. Further, a temporal resolution for use in generating attribute data is not specifically limited, as far as the temporal resolution is a period shorter than a time range of whole attendance data.

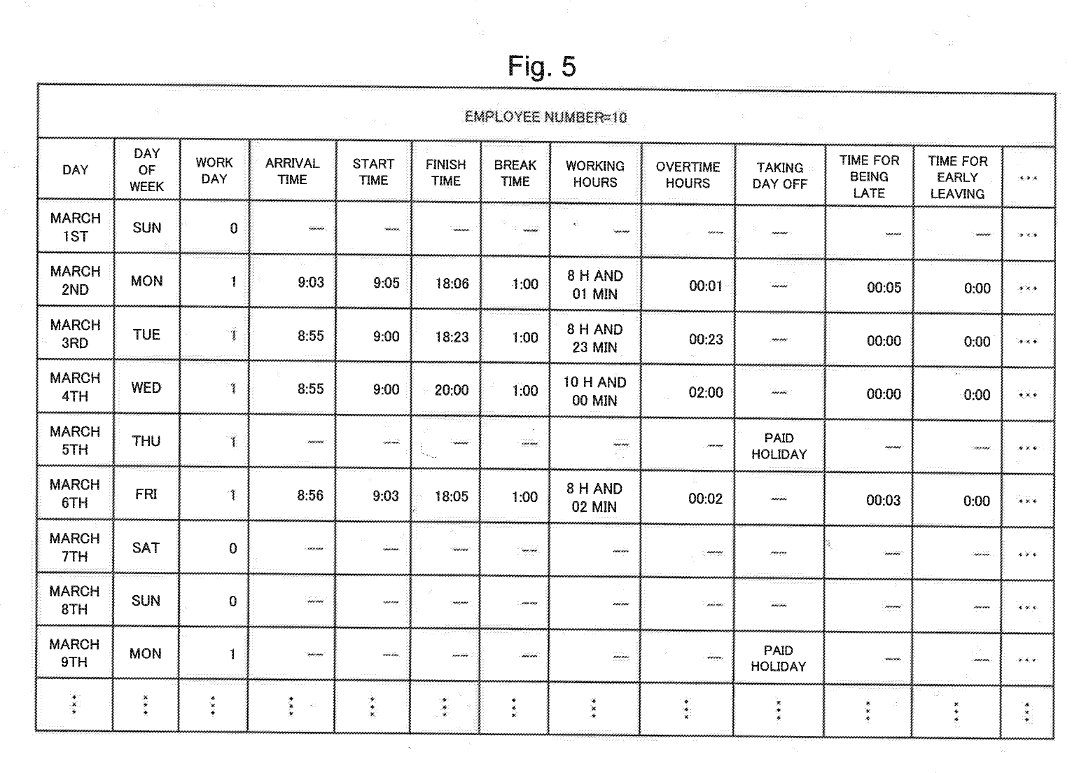

Further, FIG. 5 is an explanatory diagram illustrating a configuration example of attendance data. As illustrated in FIG. 5, attendance data may be information, in which matters relating to a work condition of each employee such as daily arrival times, daily leaving times, the presence or absence of work, the presence or absence of taking a day off, and overtime hours are arranged in a time series manner. In the present example embodiment, each matter relating to a work condition, which is included in attendance data, is referred to as a field, specifically, an attendance field. Further, a set of values of each attendance field at a certain point of time, which is included in attendance data, is referred to as a record of attendance data. Note that FIG. 5 illustrates an example of attendance data of the employee having the employee number=10. Attendance data of other employees is input in the same manner as described above.

Next, the attribute data generating unit 12 performs aggregation with respect to a predetermined field included in attendance data by using any temporal resolution, any time range, and any aggregation method; and generates attribute data (Step S12).

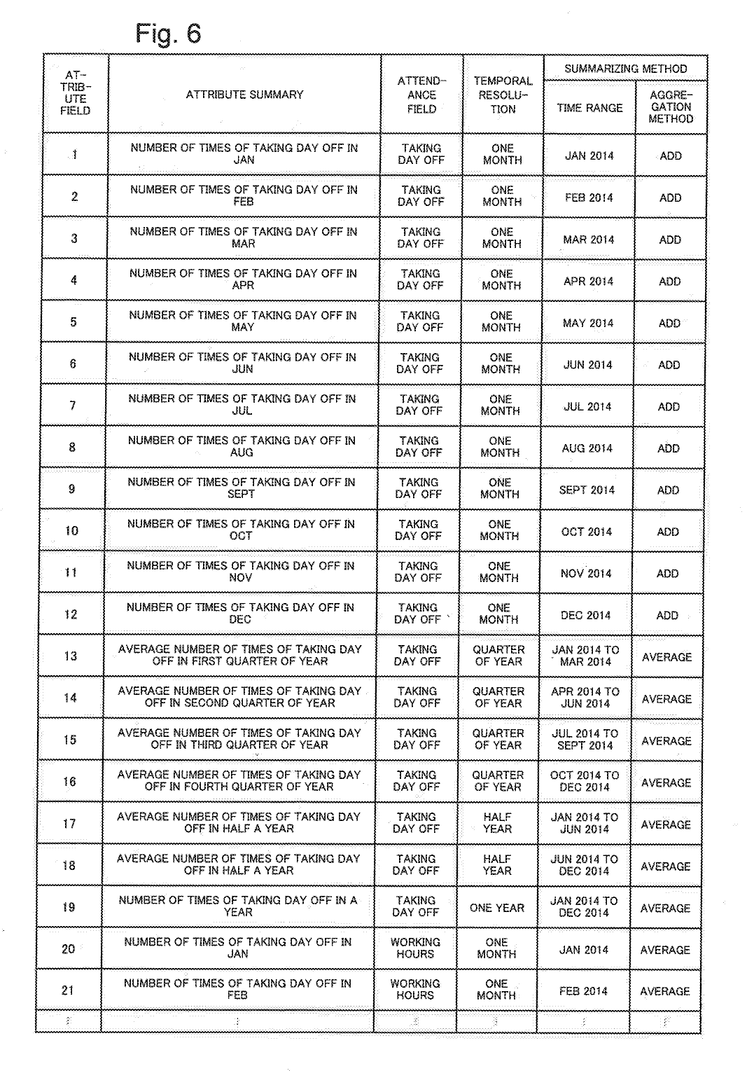

FIG. 6 is an explanatory diagram illustrating an example of attribute data setting information. The attribute data generating unit 12 may perform, as illustrated in FIG. 6, for example, aggregation processing with respect to an attendance field in accordance with attribute data setting information indicating an attribute data generation method, and may generate attribute data. FIG. 6 illustrates an example of attribute data setting information including an identifier, a summary, an attendance field for aggregation, a temporal resolution of the attendance field, a time range of the attendance field, and an aggregation method with respect to the attendance field for each of attribute fields. The attribute data generating unit 12 may perform aggregation processing with respect to a designated attendance field, based on a temporal resolution, a time range, and an aggregation method, which are indicated by such attribute data setting information, and may generate attribute data including each of aggregation results as an attribute field. Herein, a value of one attribute field may be calculated by using a plurality of attendance fields. As an example, a ratio to be calculated by using values of a plurality of attendance fields is exemplified. In this case, a plurality of attendance fields are registered in attribute data setting information, as attendance fields for aggregation.

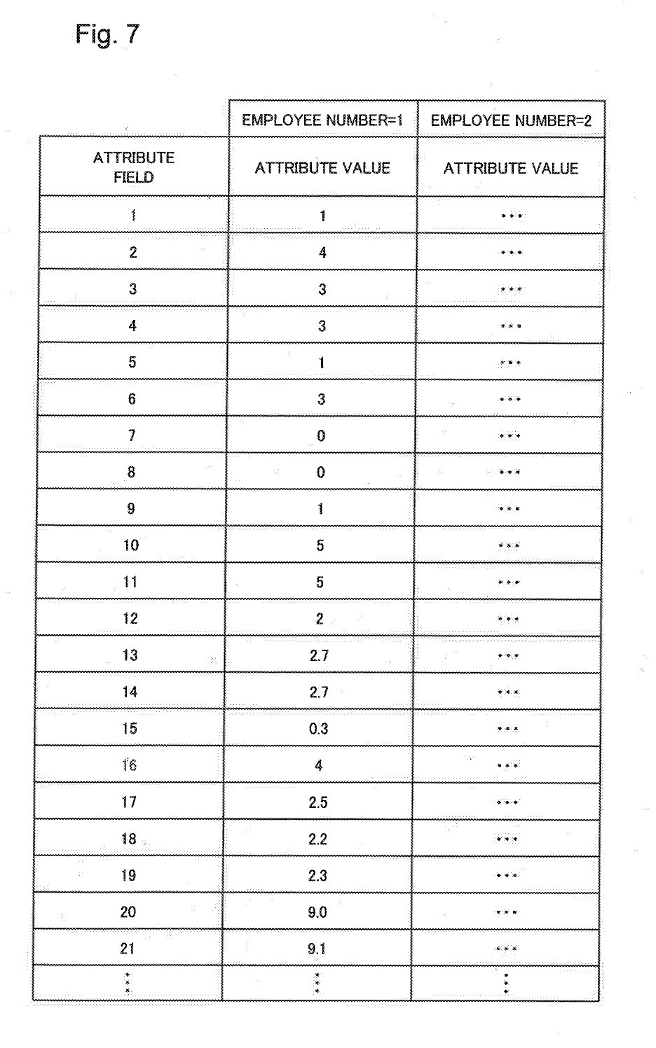

FIG. 7 is an explanatory diagram illustrating an example of attribute data. As illustrated in FIG. 7, the attribute data generating unit 12 may generate attribute data including a value of each attribute field (aggregation result) as an attribute value for each employee.

Next, the model learning unit 13 learns a model constituted by a polynomial, in which a target field is an object variable, and each of attribute fields included in attribute data generated in Step S12 is an explanatory variable, by using medical examination data (particularly, a value of a target field) and attribute data (particularly, a value of each of attribute fields) of a plurality of employees (Step S13).

Next, the related field extracting unit 14 extracts an attribute field represented by a model learned in Step S13 and associated with a target field (Step S14). Herein, the related field extracting unit 14 may extract attribute field information associated with an explanatory variable such that a model parameter (a coefficient of a polynomial) has a value other than zero, for example.

Next, the summarizing unit 15 summarizes attendance data of a designated employee, based on information extracted in Step S14, and outputs the summarized attendance data as attendance data information associated with a designated target field (Step S15). Herein, designation of employees is not limited to one person, and a plurality of employees (including all employees) may be designated. In this case, the summarizing unit 15 may summarize attendance data of each of designated employees, and may output the summarized attendance data as attendance data information associated with a designated target field.

Note that when a plurality of target fields are set, operations of Step S13 to Step S15 may be repeated for each of the target fields.

Subsequently, operations of Step S12 to Step S15 are described in more detail.

(1) More Detailed Example of Operation of Attribute Data Generating Phase (Step S12)

In the present example, it is assumed that attendance data of N employees are received. Note that N is an integer of 1 or larger. Further, attribute data of the n-th employee are expressed as X_n. Herein, n=1, . . . , N. Attribute data X_n of the present example are expressed as a vector constituted by a plurality of elements. For example, it is assumed that the number of elements (number of fields) of attribute data is seven. In this case, the attribute data generating unit 12 may generate, as attribute data of the first employee, data expressed as X_1=(0, 0, 3, 2, 1, 0, 0). This means that regarding the first employee, a value of the first attribute field is 0, a value of the second attribute field is 0, a value of the third attribute field is 3, a value of the fourth attribute field is 2, a value of the fifth attribute field is 1, a value of the sixth attribute field is 0, and a value of the seventh attribute field is 0. The attribute data generating unit 12 generates attribute data of each employee, and stores the generated attribute data in the memory 1002.

For example, in a case of the example illustrated in FIG. 6, a result of counting the number of times of taking a day off of the employee during a period from Jan. 1, 2014 to Jan. 31, 2014, more specifically, a result value obtained by summing a value of attendance field=“taking a day off” in a time range of a designated one month is received in an element (attribute value) of the first attribute field. Note that from FIG. 7, it is clear that the attribute value in attribute data of the employee: employee number=1 is 1.

Herein, one of elements (attribute fields) of attribute data of a certain employee may be the number of times of taking a day off, working hours, the number of times of taking consecutive days off, the number of times of being late, or the like which has undergone aggregation processing by using any temporal resolution regarding attendance data of the certain employee. For example, in a case of the number of times of taking a day off per month, a total number of days when the employee took a day off in the month is calculated as an attribute value of the attribute field. Further, in a case of average working hours per month, (total working hours of the month over number of work days of the month) is calculated as an attribute value of the attribute field. Note that FIG. 7 illustrates, as elements of attribute data, an example including at least the number of times of taking a day off per month, an average number of times of taking a day off in a quarter of a year, an average number of times of taking a day off in half a year, an average number of times of taking a day off in a year, and average working hours per month regarding the employee: employee number=1.

(2) More Detailed Example of Operation of Model Learning Phase (Step S13)

Hereinafter, the j-th element of attribute data of the employee n is expressed as X_nj. Herein, j=1, . . . , M (M is the number of elements of attribute data). Further, a value of a target field among medical examination data of the employee n is expressed as Y_n. The following Equation (1) is an equation expressing a relationship between Y_n and X_n.

Y_n=f(X_n) (1)

The model learning unit 13 learns a parameter necessary for expressing a function f( ) indicated by the aforementioned Equation (1). In the present example, it is assumed that f( ) is a function expressed by a polynomial constituted by an explanatory variable and a coefficient for each explanatory variable.

Herein, it is assumed that X_n is an explanatory variable in M dimensions associated with attribute data, and Y_n is a numerical value. Further, when it is assumed that W is a weight vector in M dimensions, the aforementioned Equation (1) is expressed as Equation (2). Note that one dimension for expressing a segment of a polynomial may be added to W in an M-th order vector, and an (M+1)-th order weight vector W may be set. In the following, a weight vector W is regarded as an M-th order vector, as far as the weight vector is not limited to one of an M-th order vector and an (M+1)-th order vector.

[Equation 1]

Y_n=W^{T}X_n (2)

Herein, a superscript T denotes transposition of a vector.

For example, it is assumed that a set of a value of a target field and attribute data, specifically, {X_n, Y_n} (n=1, . . . , N) is given for a plurality of employees. In this case, it is possible to calculate a value of a parameter W by optimizing an object function of the following Equation (3).

$$\begin{matrix}[ Equation 2 ] & \\ L ( W ) = ( {\sum }_{n = 1}^{N} ( Y\_n - {W}^{T} X\_n ) ) - λ ‖ W ‖ & ( 3 )\end{matrix}$$

Herein, λ is a parameter for adjusting balance between an error of a sum of squares (first term on the right side), and a penalty term (second term on the right side). Further, ∥W∥ is a norm of W. Normally, L1 norm or L2 norm is used. Further, L(W) is a convex function relating to W. It is possible to maximize L(W) by a method pursuant to a gradient method.

The model learning unit 13 may obtain a value of a parameter W which maximizes L(W) of the aforementioned Equation (3), as model learning processing, for example. Hereinafter, a value of a parameter W obtained herein may be expressed as W_{c}. The model learning unit 13 stores an obtained W_{c} in the memory 1002.

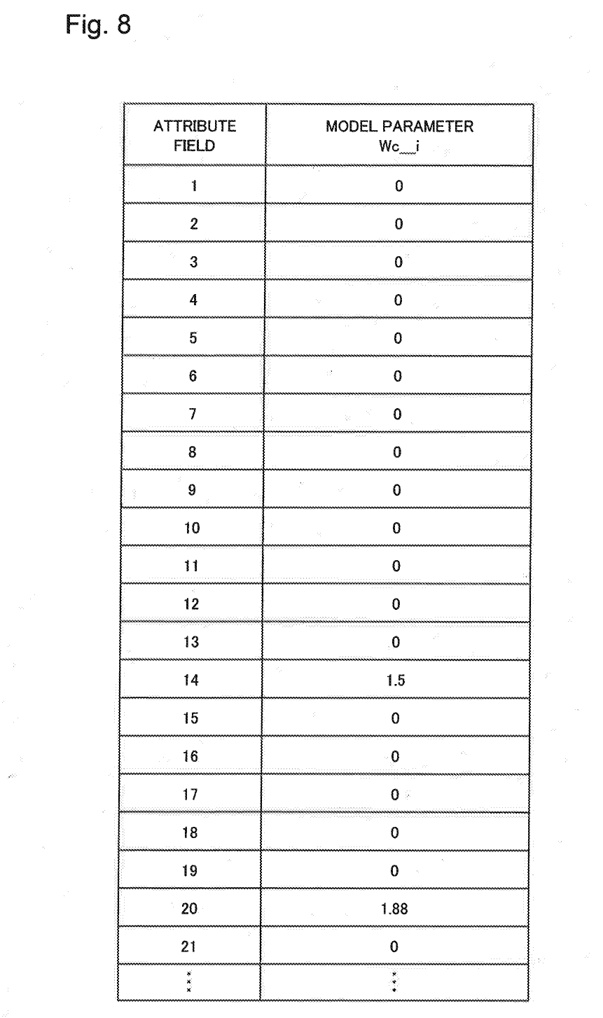

FIG. 8 is an explanatory diagram illustrating an example of an attribute table including a model parameter W_{c} obtained as a result of learning. FIG. 8 illustrates an example, in which parameters W_{c}_{_}14 and W_{c}_{_}20 corresponding to coefficients of the 14-th and 20-th attribute fields have values other than zero, and parameters W_{c}_{_}1 to W_{c}_{_}13, W_{c}_{_}15 to W_{c}_{_}19, and W_{c}_{_}21 and thereafter other than the aforementioned parameters have a value zero. The model learning unit 13 may store, in the memory 1002, an attribute table, in which an identifier of an attribute field and a parameter W_{c}_{_}j obtained as a coefficient of the attribute field are associated with each other, as illustrated in FIG. 8, for example.

(3) More Detailed Example of Operation of Related Field Extracting Phase (Step S14)

The related field extracting unit 14 reads, from an attribute table stored in the memory 1002, a value of each model parameter W_{c}_{_}j (j=1, . . . , M) corresponding to a coefficient of a polynomial, for example.

Further, the related field extracting unit 14 may extract an identifier of an attribute field associated with W_{c}_{_}j having a value other than zero among read W_{c}_{_}j. Further, the related field extracting unit 14 may extract a set of an attendance field, a temporal resolution, a time range, and an aggregation method used in generating the attribute field, based on an extracted identifier.

For example, the related field extracting unit 14 may extract a set of an attendance field used in generating the j-th attribute field, and a temporal resolution, a time range, and an aggregation method with respect to the attendance field, based on attribute data setting information, regarding j having a value of |W_{c}_{_}j| being an absolute value larger than zero among W_{c}_{_}j (j=1, . . . , M).

Herein, a case where W_{c}_{_}j has a negatives value means that there is a negative correlation between a target field and the j-th attribute field. Further, a case where W_{c}_{_}j has a positive value means that there is a positive correlation between a target field and the j-th attribute field. Note that a case where W_{c}_{_}j is zero means that there is no correlation between a target field and the j-th attribute field.

The related field extracting unit 14 may extract a set of an attendance field, and a temporal resolution, a time range, and an aggregation method with respect to the attendance field regarding all attribute fields associated with W_{c}_{_}j having a value other than zero, as a result of model learning by the model learning unit 13. Further, the related field extracting unit 14 may store extracted information in the memory 1002.

For example, in a case of an example of the attribute table illustrated in FIG. 8, since parameters W_{c}_{_}14 and W_{c}_{_}20 corresponding to coefficients of the 14-th and 20-th attribute fields have values other than zero, regarding the 14-th and 20-th attribute fields, a set of an attendance field, and a temporal resolution, a time range, and an aggregation method with respect to the attendance field is extracted and stored in the memory 1002.

(4) More Detailed Example of Operation of Summarizing Phase (Step S15)

The summarizing unit 15 reads, from the memory 1002, a set of an attendance field, a temporal resolution, a time range, and an aggregation method, which is stored as attribute field information associated with a target field. Further, the summarizing unit 15 summarized attendance data of a designated employee, based on read information, and outputs a result of the summarization. An output destination may be the memory 1002, the output device 1003, another device to be connected via the network interface 1005, or the like.

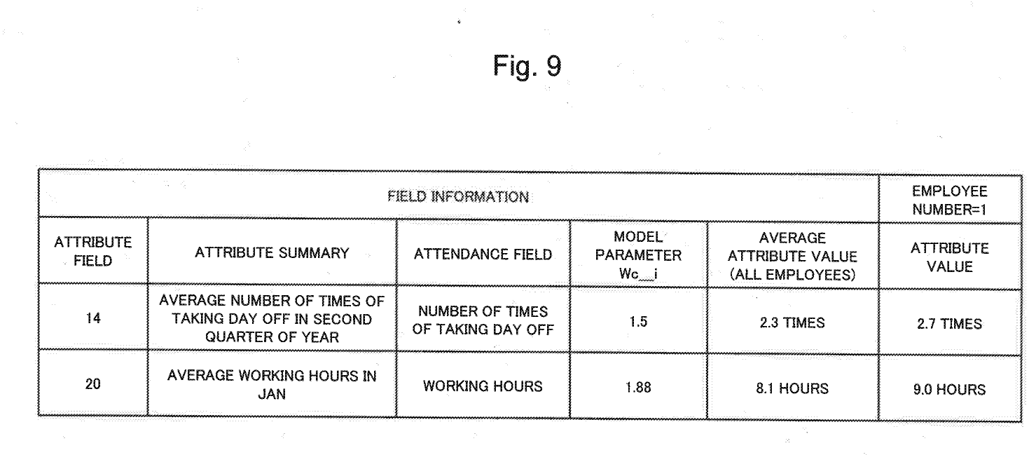

FIG. 9 is an explanatory diagram illustrating an example of a summary result of attendance data to be output by the summarizing unit 15. As illustrated in FIG. 9, the summarizing unit 15 may output an attribute value of a designated employee together with a summary of the attribute field, an attendance field used in generation, and a degree of positive/negative correlation, regarding all attribute fields having a positive or negative correlation to a target field. Herein, the attribute value corresponds to a summary result of attendance data of the employee. Further, a model parameter W_{c}_{_}j corresponds to information indicating a degree of positive/negative correlation. Note that FIG. 9 illustrates an example, in which an average of the attribute values of all employees is also output in addition to the aforementioned information. Further, although illustration is omitted in FIG. 9, information on a summarizing method (such as a temporal resolution, a time range, and an aggregation method) may also be output.

By outputting an average of attributes values of all employees, for example, an advisor is able to easily comprehend whether an attribute value of an employee to be guided (in this case, employee: employee number=1) is larger or smaller than attribute values of other employees. This is helpful in health guidance.

For example, in the example illustrated in FIG. 9, regarding the employee: employee number=1, an average number of times of taking a day off in the second quarter of a year during an attendance data collection period is 2.7 times, which is larger than an average number of times of all employees, i.e., 2.3 times. Further, from a value of a model parameter W_{c}_{_}j being a coefficient of the attribute field, it is clear that an attribute value of the attribute field, specifically, an average number of times of taking a day off in the second quarter of a year has a positive correlation to a target field. This can be interpreted that the greater the attribute value is, the greater a value of a target field is. As a concrete example, for example, when it is assumed that a target field is a blood glucose level, the greater an attribute value of the attribute field is, the greater a value of the blood glucose level is. An advisor may point out that an average number of times of taking a day off in the second quarter of a year is large, as one of factors that a value of a target field of the employee is high, for example. Note that the same judgement as described above is also given regarding average working hours in January.

As described above, a correlation between a target field and attendance data is easily and concretely comprehended. Therefore, an advisor is able to provide an appropriate advice. According to the aforementioned example, an advisor is able to provide an advice relating to a work condition to the employee: employee number=1 from an aspect of health promotion by focusing on an average number of times of taking a day off in the second quarter of a year and average working hours in January.

As described above, the present example embodiment is not only able to present an attendance field associated with any designated medical examination field, but also able to provide accurate information on a temporal resolution, a time range, an aggregation method, and the like with respect to the attendance field, and attendance data which are actually summarized by these methods, for an advisor. Therefore, an advisor is able to provide an appropriate advice, based on these pieces of information. Further, an advisor is not only able to summarize and present attendance data, but also able to provide what relevance, an attendance field included in the summarized attendance data has with respect to a target field, and a degree thereof (degree of positive or negative correlation). Therefore, an advisor is able to provide a more appropriate advice, based on these pieces of information.

Next, some modification examples of the present example embodiment are described.

### First Modification Example

The data analysis device illustrated in FIG. 1 is made for the purpose of allowing an advisor to easily grasp/comprehend, from attendance data, the presence or absence of a work condition associated with health conditions of an employee at a determination time, and the like. In view of the above, the data analysis device expresses relevance between medical examination data before a determination time, and attendance data for a predetermined period earlier than a medical examination day when the medical examination data are obtained by coefficients of a polynomial model, and summarizes and outputs attendance data of each employee for the aforementioned predetermined period, based on a value of each coefficient to be obtained by learning the model.

On the other hand, it is also important for an advisor to perform not only health guidance for the purpose of maintaining/promoting health conditions of an employee at a determination time, but also health guidance for health promotion in the future at an early stage for the purpose of maintaining/promoting health conditions of the employee in the future such as half a year after, one year after, or three years after when medical examination data are not obtained, for example.

In view of the above, in the first modification example, it is possible to output attendance data information associated with a target field at a future point of time and already acquired at a current point of time.

More specifically, the following information is added to input information. That is, first attendance data for use in learning, and second attendance data for use in presenting relevance to a target field at a future point of time are received as attendance data.

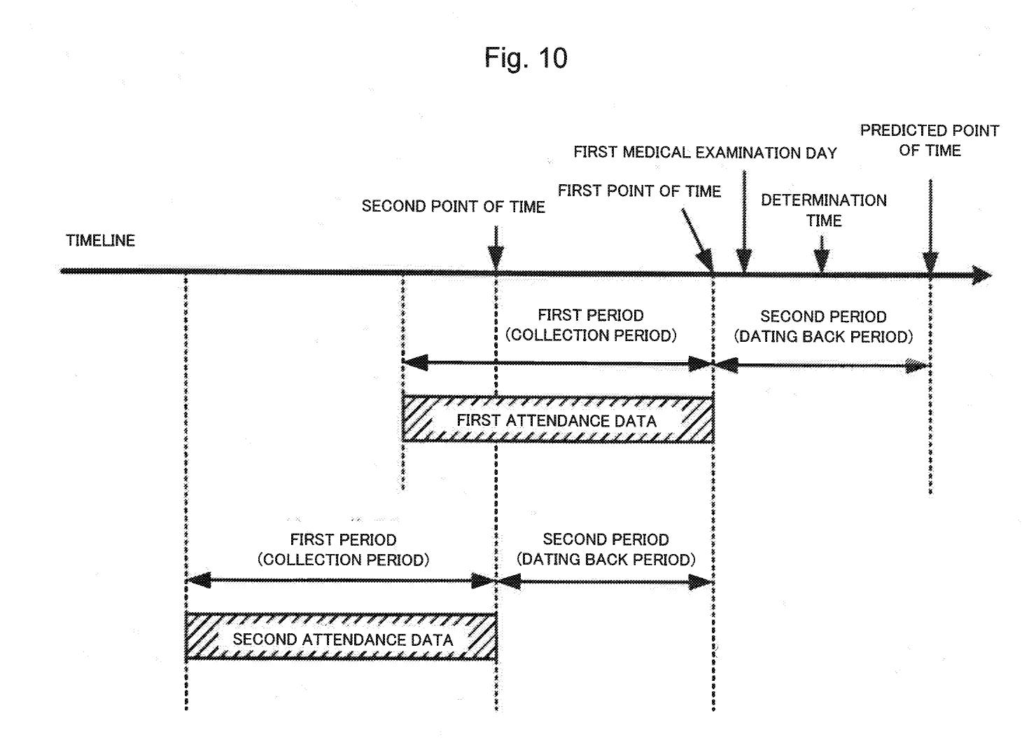

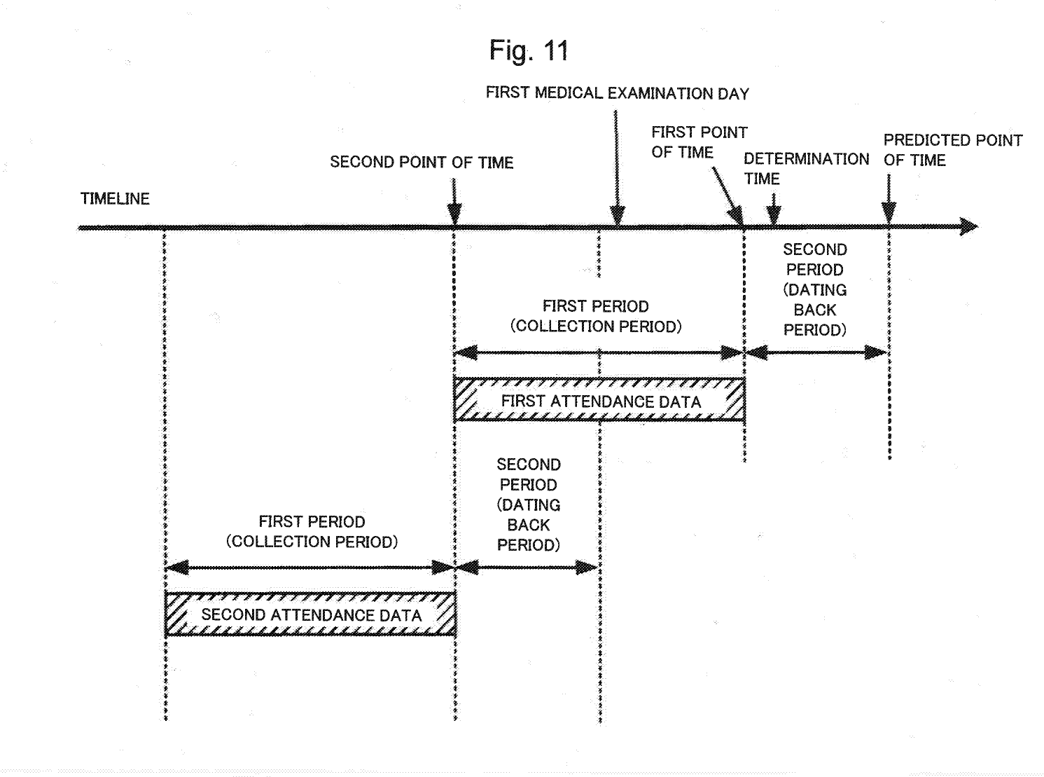

FIG. 10 is an explanatory diagram illustrating an example of attendance data to be received in the first modification example. As illustrated in FIG. 10, a data input unit 11 in the present example may receive, for example, as attendance data, first attendance data including records for a first period before a predetermined first point of time earlier than a determination time, and second attendance data including records for a first period before a second point of time being a predetermined point of time dating back from a latest medical examination day (first medical examination day) by a predetermined second period or longer. Note that in the example illustrated in FIG. 10, the first medical examination day is illustrated as a day later than the first point of time. A relationship between the first medical examination day and the first point of time is not limited to the above. Specifically, the first medical examination day may be earlier than the first point of time (see FIG. 11 to be described later).

In the present example, learning is performed by using a content of a target field of medical examination data on a first medical examination day as an object variable, and by using a content of each of attribute fields of second attribute data to be generated by using second attendance data as an explanatory variable. Further, relevance between a content of each piece of first attribute data to be generated by using first attendance data, and a content of a target field at a predicted point of time being a future point of time is presented, based on a learned content. In other words, a first period before a second point of time is set as a period for use in learning, and a first period before a first point of time is set as a period for use in prediction. More specifically, first attendance data are used as an object from which relevance to a target field at a predicted point of time is derived, that is, as attendance data for use in prediction; and second attendance data are used as attendance data for use in learning for prediction.

Further, FIG. 11 is an explanatory diagram illustrating another example of attendance data to be received in the first modification example. As illustrated in FIG. 11, for example, a data input unit 11 may receive first attendance data including records for a first period before a first point of time by assuming that a predetermined point of time dating back from a predicted point of time by a second period or longer is the first point of time, and may receive second attendance data including records for a first period before a second point of time by assuming that a predetermined point of time dating back from a latest medical examination day (first medical examination day in FIG. 11) earlier than a determination time by a second period or longer is the second point of time. In this case, the predicted point of time may be a future point of time later than any first point of time earlier than a determination time by a second period. Note that in the present example, the first point of time may be any point of time earlier than a determination time, and may not necessarily be earlier than a first medical examination day. Further, the second point of time may be any day dating back from a first medical examination day earlier than a determination time by a second period or longer. Note that a first attendance data collection period and a second attendance data collection period may not necessarily be consecutive, or may not necessarily overlap each other. Specifically, any number of days may be provided between a first attendance data collection period and a second attendance data collection period.

Hereinafter, latest medical examination data before a determination time may be referred to as first medical examination data. Further, hereinafter, the first period may be referred to as a collection period, and the second period may be referred to as a dating back period. Note that the second period may be optionally set, as far as a period between a first medical examination day and a second point of time is a certain period or longer, more specifically, is a period equal to or longer than a period from a first point of time to an intended predicted point of time. It does not particularly matter whether the second period is longer or shorter than the first period. Specifically, the second period may be equal to the first period, or may be shorter or longer than the first period. Note that first attendance data and second attendance data are not specifically discriminated. One piece of attendance data including records for a period including both periods, i.e., a first attendance data collection period and a second attendance data collection period may be received. Even in such a case, in the following, for convenience of explanation, first attendance data and second attendance data are expressed in a discriminated manner.

A configuration of the present modification example is basically the same as the configuration of the first example embodiment illustrated in FIG. 1.

In the present modification example, the data input unit 11 receives second attendance data of each employee, in addition to input information in the aforementioned first example embodiment.

Further, an attribute data generating unit 12 generates attribute data, based on second attendance data received of each employee. Note that an attribute data generation method may be the same as in the first example embodiment. Hereinafter, attribute data to be generated by using second attendance data may be referred to as second attribute data, and attribute data to be generated by using first attendance data may be referred to as first attribute data. The attribute data generating unit 12 may generate first attribute data, in addition to second attribute data.

Note that in the example of the attribute data setting information illustrated in FIG. 6, a time range is illustrated in terms of concrete dates or the like. However, in a time range of attribute data setting information in the present example, it is assumed that a content such as “data for January of the year at a start time” is set, based on a point of time when attendance data for aggregation are collected (e.g. a point of time dating back from a second medical examination day by a first period).

Further, a model learning unit 13 learns a polynomial model, in which a target field of first medical examination data is an object variable, and each of attribute fields of second attribute data is an explanatory variable by using first medical examination data and second attribute data of a plurality of employees. Note that the present example is different from the aforementioned first example embodiment in a point that second attribute data are used, in place of first attribute data. The model is said to be a model representing an influence of attendance data before a point of time (second point of time) dating back from a first medical examination day by a second period or longer, on a value of a target field to be acquired on the first medical examination day.

A related field extracting unit 14 may be the same as in the aforementioned first example embodiment. Specifically, the related field extracting unit 14 extracts an attribute field represented by a learned model and associated with a target field.

A summarizing unit 15 summarizes and outputs first attendance data, based on information extracted by the related field extracting unit 14, for example. Summarizing processing may be the same as in the first example embodiment. The summarizing unit 15 may output, regarding all attribute fields in which a correlation to a target field is recognized by model learning, an attribute value of first attribute data of a designated employee together with information on the attribute field (such as an attendance field, a summarizing method, and a degree of relevance), for example. Further, the summarizing unit 15 is also able to use an attribute value of first attribute data by omitting summarizing processing, when first attribute data are already generated.

According to the aforementioned configuration, an advisor is able to obtain attendance data information associated with a target field at a predicted point of time. An advisor is able to easily grasp/comprehend the presence or absence of a work condition or the like, which is predicted to affect a value of a target field of medical examination data at a point of time later than a first medication examination day by a second period or longer (e.g. half a year after or one year after) for each employee, based on first attendance data summarized as described above, for example.

Herein, relevance between a target field of first medical examination data and second attendance data, which is indicated by information extracted by the related field extracting unit 14, is obtained by using data earlier than a determination time, more specifically, by using medical examination data on a first medical examination day as an object variable, and by using each of attribute fields to be generated from second attendance data collectable at a second point of time dating back from the first medical examination day by a second period or longer as an explanatory variable. Therefore, relevance between medical examination data at a predicted point of time in the future and first attendance data collectable at a first point of time dating back from the predicted point of time in the future by a second period or longer is not directly obtained for each employee. However, in the present modification example, it is assumed that there is no great change generated between relevance between a value of a target field on a first medical examination day earlier than a determination time and second attendance data; and relevance between a value of a target field at a predicted point of time later than the determination time, and first attendance data. Thus, an advisor is able to easily grasp/comprehend the presence or absence of overwork or irregular work shifts which is associated with a target field on a medical examination day in the future serving as a predicted point of time for any employee, based on first attendance data summarized by a summarizing method to be specified by a model which is learned by using second attribute data to be generated from second attendance data, as learning data.

### Second Modification Example

In the present modification example, a predicted value of a target field of medical examination data at a future point of time is further provided to an advisor, in addition to functions of the first modification example.

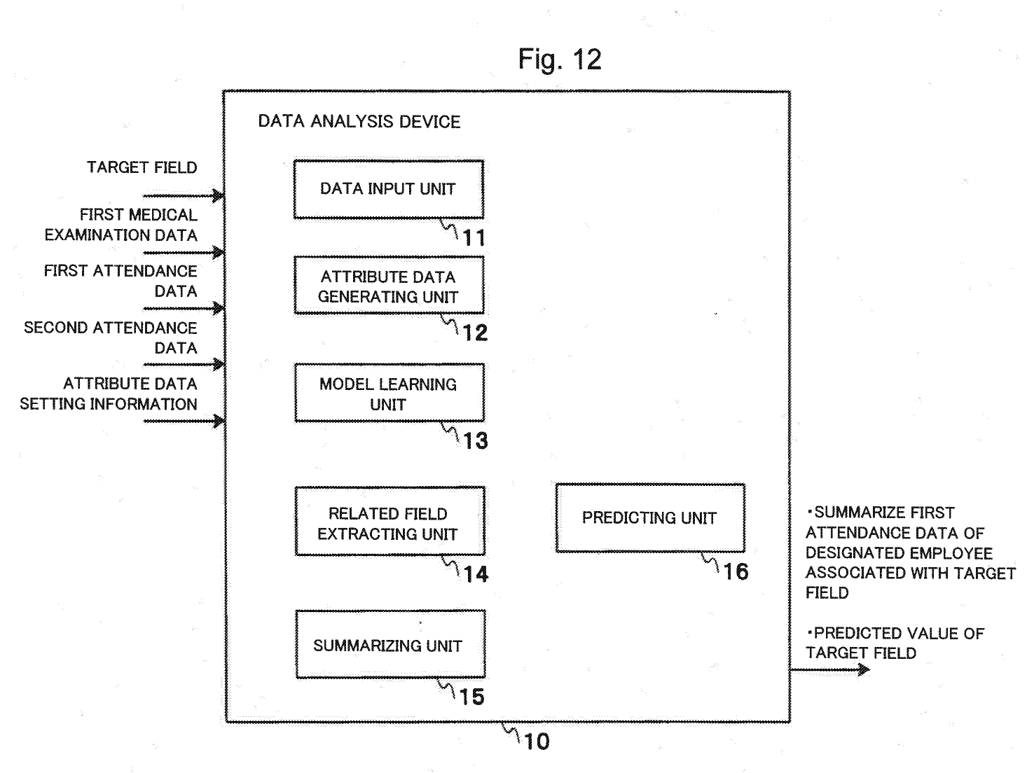

FIG. 12 is a block diagram illustrating a configuration example of a data analysis device of the present modification example. The data analysis device 10 illustrated in FIG. 12 further includes a predicting unit 16, in addition to the configuration of the first modification example.

Note that a data input unit 11, an attribute data generating unit 12, a model learning unit 13, and a related field extracting unit 14 may be the same as in the first modification example.

The predicting unit 16 predicts a value of a target field at a predetermined predicted point of time by using a learned model, and first attribute data of a designated employee.

For example, the predicting unit 16 may calculate a value of a target field at a predicted point of time by the following Equation (4) by using a parameter W_{c} of a learned model, and first attribute data. Note that in the present modification example, first attribute data of a designated employee for use in prediction are expressed as X′_n. Herein, the predicted point of time may be a latest medical examination day later than a first medical examination day by a second period or longer.

Y′_n=W_{c}^{T}·X′_n (4)

The predicting unit 16 stores calculated Y′_n in a memory 1002. Herein, Y′_n denotes a predicted value of a target field at a predicted point of time for the employee n.

A summarizing unit 15 further outputs a predicted value of a target field predicted by the predicting unit 16, in addition to functions of the summarizing unit 15 in the first modification example, for example.

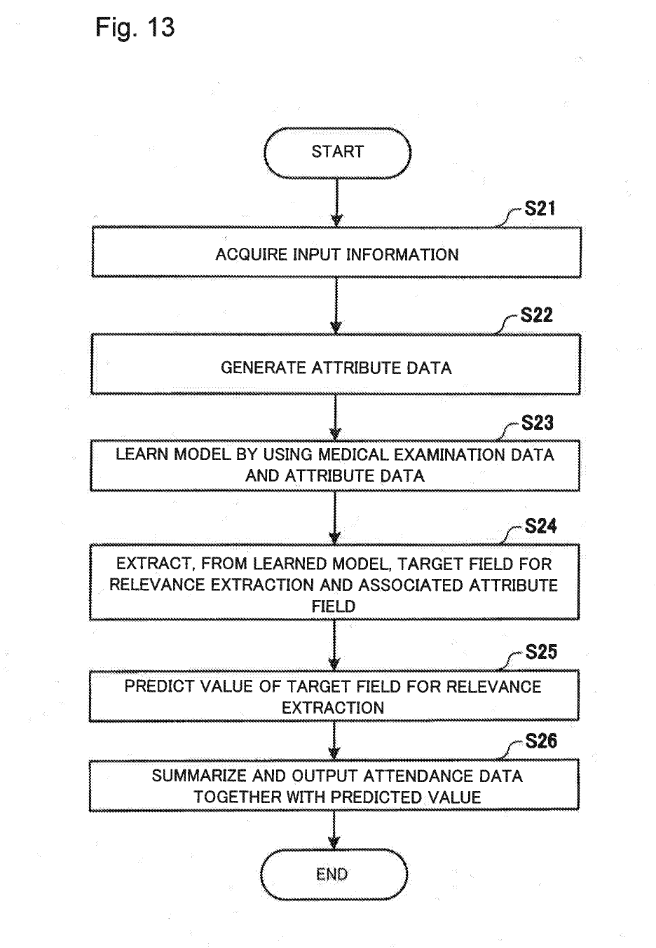

FIG. 13 is a flowchart illustrating an example of an operation of the data analysis device of the present modification example. In the example illustrated in FIG. 13, first of all, the data input unit 11 receives necessary information (Step S21). In the present example, the data input unit 11 receives designation of a target field, first medical examination data, first attendance data, and second attendance data of each employee.

Subsequently, the attribute data generating unit 12 generates second attribute data, based on second attendance data (Step S22).

Subsequently, the model learning unit 13 learns a model by using a value of a target field of first medical examination data, and a content of second attribute data of a plurality of employees (Step S23).

Subsequently, the related field extracting unit 14 extracts attribute field information represented by the learned model and associated with a target field (Step S24).

Subsequently, the predicting unit 16 calculates a predicted value of a target field of a designated employee at a predicted point of time by using the learned model, and first attribute data of the designated employee (Step S25).

Lastly, the summarizing unit 15 summarizes first attendance data of the designated employee, based on information extracted in Step S24, and outputs a predicted value calculated in Step S25 together with a summary result (Step S26).

This allows an advisor to provide an advice for health promotion to any focused employee, based on a determination as to whether a medical examination result in the future is good or bad, while grasping/comprehending the presence or absence of overwork or irregular work shifts at a current stage, which is associated with the medical examination result in the future of the employee, or the like.

For example, it is assumed that a longer time for health guidance is secured, or a more strict advice for improving a work condition is provided to an employee having a predicted medical examination value in the future in an abnormal range in order to provide improvements on overwork or irregular work shifts, which is associated with the item at a predicted point of time.

### Third Modification Example

In the present modification example, medical examination data are also used in addition to attendance data when attribute data are generated.

An attribute data generating unit 12 may include, for example, in an attribute field of attribute data, a result obtained by performing aggregation processing with respect to a value of a predetermined medical examination field of medical examination data, for example, a blood pressure, a blood glucose level (such as HbA1c), lipid (such as HDL and LDL), a height, a body weight, a value of an interview result (such as answers to questions relating to smoking habits, sleep habits, and meal habits) by using a predetermined method.

For example, in the aforementioned example embodiment and in each of the modification examples, the attribute data generating unit 12 may set X_nj (j=1, . . . , M+K) by including a medical examination field of medical examination data of the employee n. Herein, K denotes the number of medical examination fields to be added to X_nj. Note that a target field is not included in K. Note that when relevance to a target field at a future point of time is obtained, a target field of existing medical examination data may be included in K. Hereinafter, a target field of medical examination data, from which relevance is actually extracted, is referred to as a “target field”.

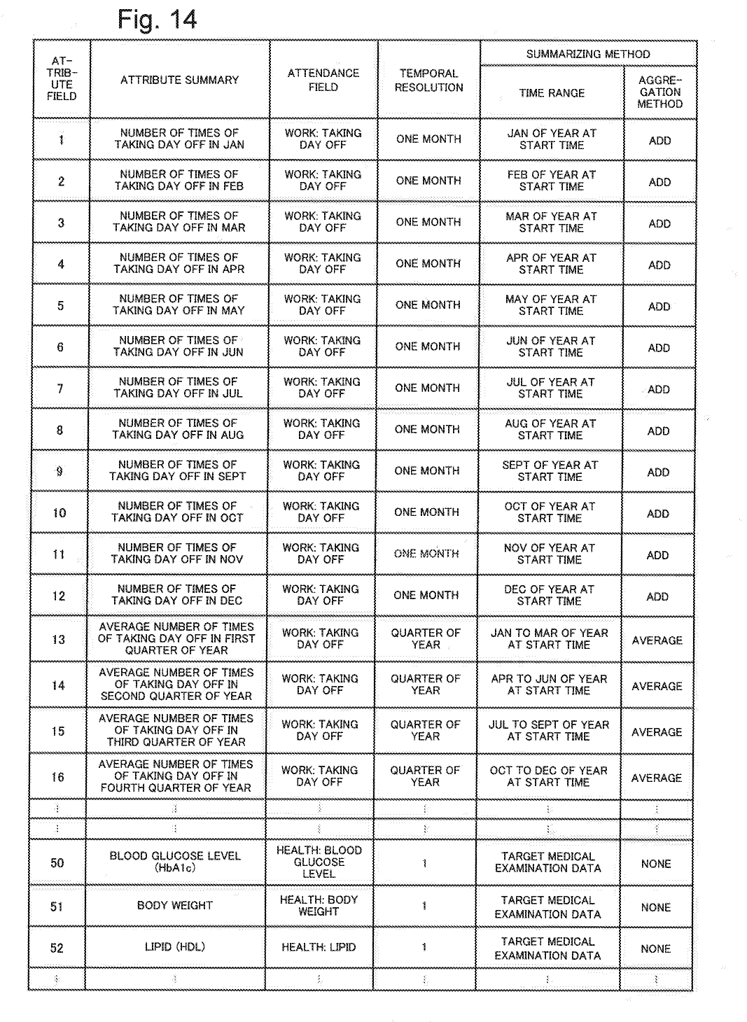

FIG. 14 is an explanatory diagram illustrating an example of attribute data setting information in the present modification example. As illustrated in FIG. 14, the attribute data generating unit 12 may store in advance attribute data setting information indicating an attribute data generation method, as input information including medical examination data in addition to attendance data, for example. FIG. 14 illustrates an example, in which values of blood glucose level (HbA1c), body weight, and lipid (HDL) are used as elements of attribute data, specifically, as attribute fields among fields of medical examination data.

Note that in the example illustrated in FIG. 14, it is possible to designate medical examination result data in addition to attendance data, as a data field. For example, in FIG. 14, a data field=“work_taking a day off” denotes that a data field for aggregation is a field of taking a day off in attendance data. Further, for example, a data field=“health_blood glucose level” denotes that a data field for aggregation is a field of blood glucose level in medical examination data. In addition, an aggregation method=“none” denotes that a value as it is used.

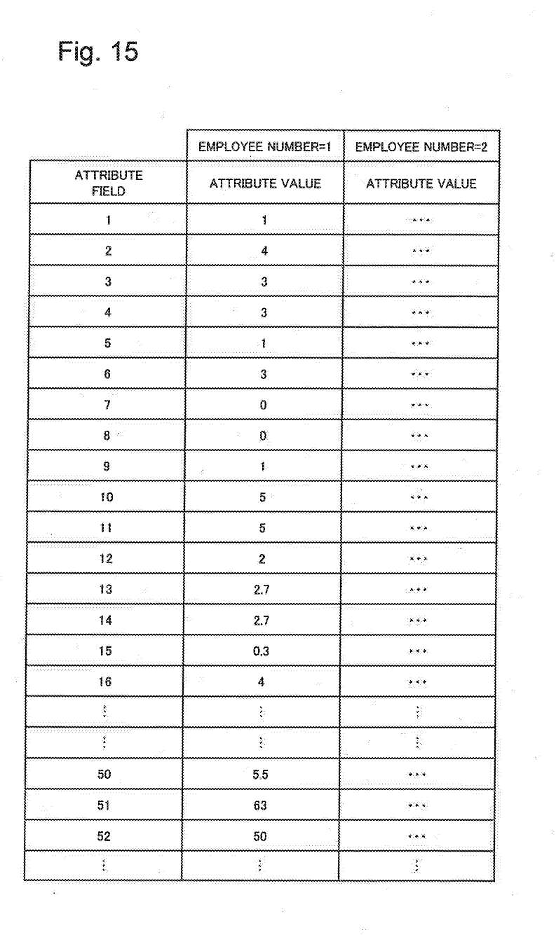

Further, FIG. 15 is an explanatory diagram illustrating an example of attribute data to be generated based on the attribute data setting information illustrated in FIG. 14. In the example illustrated in FIG. 15, at least values of 50-th to 52-nd attribute fields are set as values of a medical examination field.

Note that as the number of attribute fields increases, the number of model parameters W_j increases.

For example, it is assumed that the present modification example is combined with the aforementioned first example embodiment. In this case, an attribute data generating unit 12 generates attribute data of each employee from attendance data received and medical examination data received, based on attribute data setting information.

Further, a related field extracting unit 14 may extract, as attribute field information represented by a learned model and associated with a target field, information relating to at least one of an identifier of an attendance field and a medical examination field used in generation, and a summary, or the like.

Further, a summarizing unit 15 summarizes and outputs attendance data and medical examination data, based on information extracted by the related field extracting unit 14.

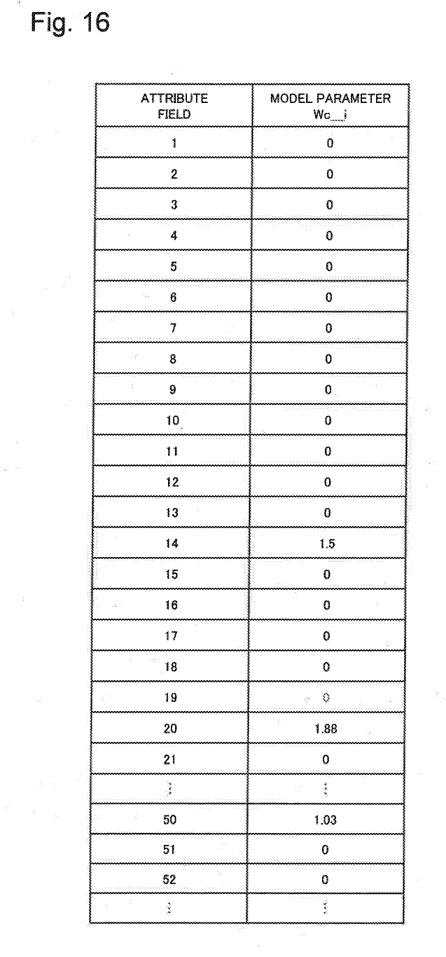

FIG. 16 is an explanatory diagram illustrating another example of an attribute table. From FIG. 16, it is clear that parameters W_{c}_{_}14, W_{c}_{_}20, and W_{c}_{_}50 corresponding to coefficients of the 14-th, 20-th, and 50-th attribute fields have values other than zero, as a result of model learning.

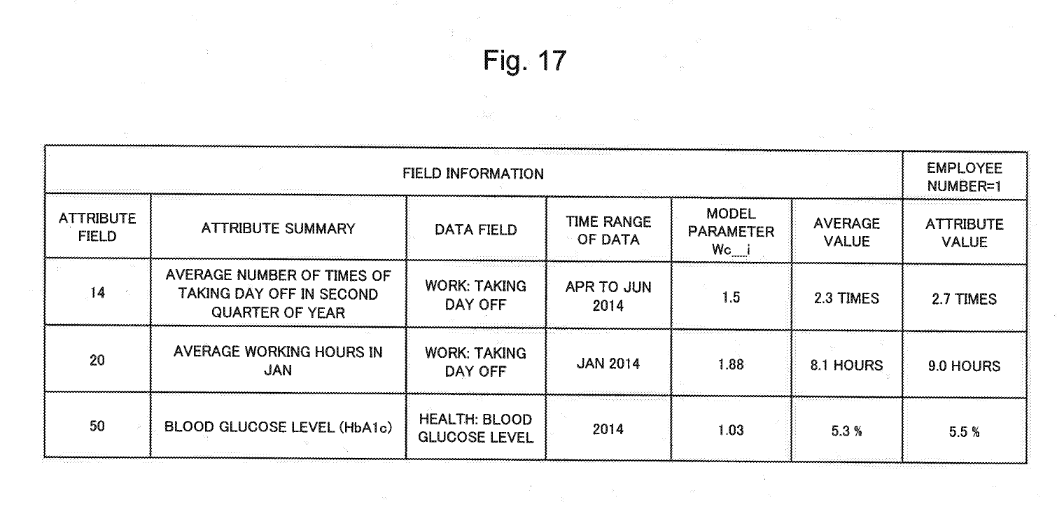

Further, FIG. 17 is an explanatory diagram illustrating an example of a summary result of attendance data and medical examination data to be output by the summarizing unit 15. As illustrated in FIG. 17, a summary result may include an identifier of an attribute field, a summary, a field name of original attendance data or medical examination data, a time range, a degree of relevance (model parameter W_{c}_{_}j), an average value, and an aggregation result (attribute value). In addition to the above, a summary result may further include information on a temporal resolution and an aggregation method.

Further, for example, it is assumed that the present modification example is combined with the aforementioned second modification example. In this case, a data input unit 11 receives second medical examination data included in a second attendance data collection period or collected within a predetermined number of days from the collection period (e.g. until a point of time when a predetermined number of days elapse), in addition to designation of a target field, and first medical examination data, first attendance data, and the second attendance data of each employee.

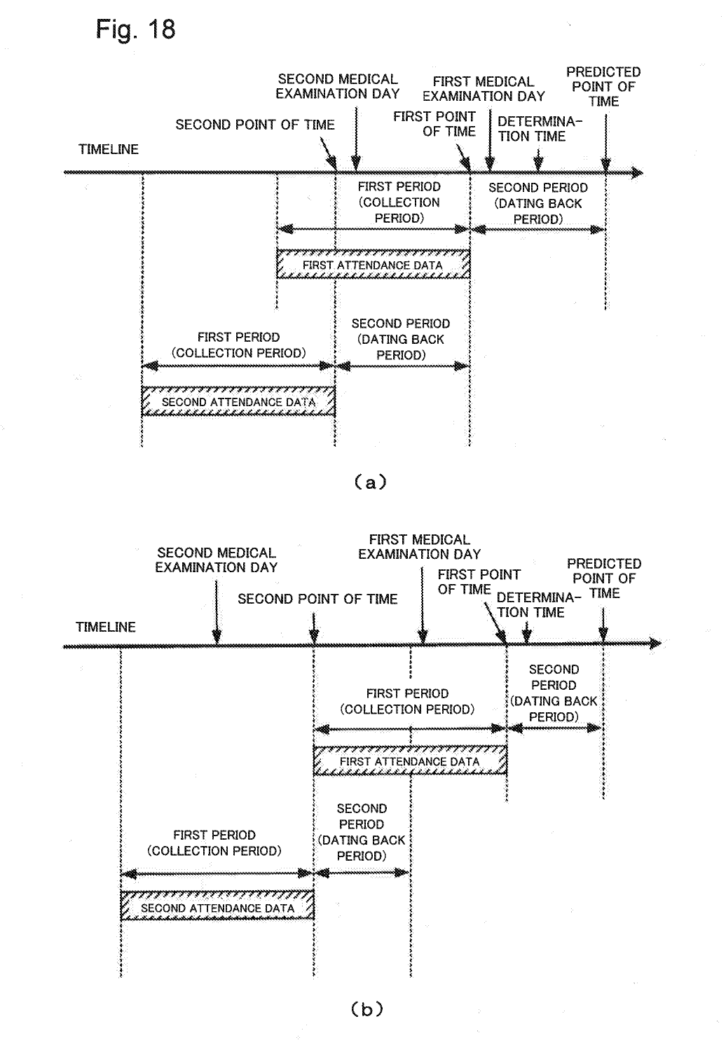

FIG. 18 is an explanatory diagram illustrating a relationship between attendance data and medical examination data (more specifically, a medical examination day) in the present modification example. As illustrated in FIG. 18(a), a data input unit 11 may receive, as first medical examination data, medical examination data on a first medical examination day by assuming that a latest medical examination day later than a last day of a first attendance data collection period is the first medical examination day; and may receive, as second medical examination data, medical examination data on a second medical examination day by assuming that a latest medical examination day later than a last day of a second attendance data collection period is the second medical examination day, for example. Further, as illustrated in FIG. 18(b), for example, the data input unit 11 may receive, as first medical examination data, medical examination data on a first medical examination day by assuming that a medical examination day within a first attendance data collection period is the first medical examination day, and may receive, as second medical examination data, medical examination data on a second medical examination day by assuming that a medical examination day within a second attendance data collection period is the second medical examination day, for example.

An attribute data generating unit 12 generates second attribute data of each employee from second attendance data received and second medical examination data received, based on attribute data setting information. Further, the attribute data generating unit 12 may further generate first attribute data of each employee from first attendance data received and first medical examination data received.

A model learning unit 13 learns a model, in which a target field included in first medical examination data is an object variable, and a value of the object variable is calculated by using second attribute data.

A related field extracting unit 14 may extract, as attribute field information represented by a learned model and associated with a target field, information relating to at least one of an identifier of an attendance field and a medical examination field used in generation, and a summary, or the like.

A predicting unit 16 predicts a value of a target field at a predicted point of time by using a learned model, and first attribute data of a designated employee.

A summarizing unit 15 summarizes first attendance data and first medical examination data, based on information extracted by the related field extracting unit 14, and outputs a predicted value of a target field together with a summary result.

According to the present modification example, an advisor is not only able to easily grasp/comprehend the presence or absence of overwork, irregular work shifts, or the like, which is associated with any item relating to a focused health condition, but also able to easily grasp/comprehend another inspection value, or the presence or absence of an interview result associated with the item, or the like. This makes it possible to provide further efficient health guidance.

### Fourth Modification Example

Next, the fourth modification example is described. In health guidance, it is required to provide an appropriate advance depending on characteristics of each employee, taking into consideration occupations of employees and features of each office. For example, among employees having different occupations or working in different offices, arrival times may be different, break times may be different, or average overtime hours may be different.

In view of the above, employees may be classified into groups, and processing of the aforementioned example embodiment or each of the modification examples may be performed for each group by taking into consideration a difference of occupations of employees, a difference of offices, or the like.

Specifically, in the present modification example, processing of learning a model for each group, and extracting attribute field information associated with a target field is performed. Further, when summarization is performed, attendance data, and medical examination data as necessary are summarized, based on attribute field information extracted for each group to which a designated employee belongs, or the like. In addition, also when a predicted value of a target field is calculated, calculation is performed by using a model for each group to which a designated employee belongs.

As a method for classifying employees into groups, determination may be made in advance such that to which group each employee belongs, or employees may be classified into groups based on a predetermined condition. Further, employees may be classified into groups based on attribute data generated by an attribute data generating unit 12. In addition, it is also possible to classify employees into groups based on medical examination data.

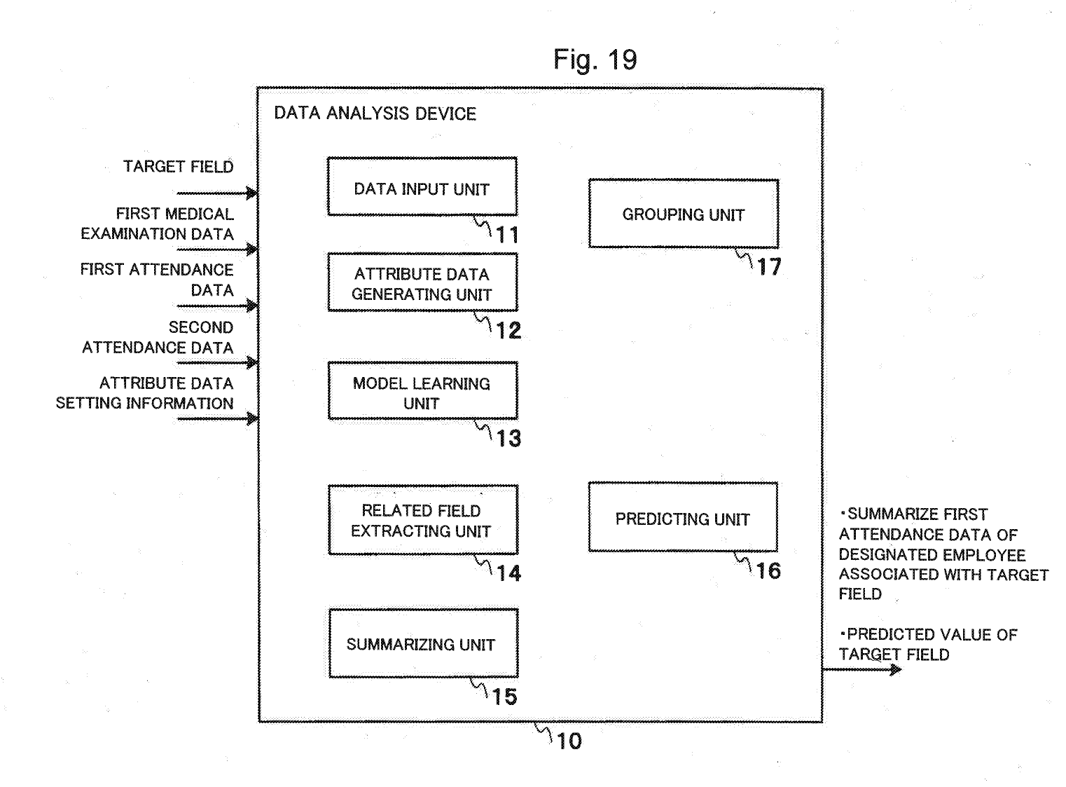

FIG. 19 is a block diagram illustrating a configuration example of a data analysis device in the present modification example. FIG. 19 illustrates a configuration example, in which the fourth modification example is combined with the third modification example. The data analysis device 10 illustrated in FIG. 19 further includes a grouping unit 17, in addition to the configuration of the third modification example.

When employees are classified into groups based on a predetermined condition, for example, the grouping unit 17 may classify employees having a same or similar content into a same group by using an item such as an office, a department, an occupation, a generation, the sex, or the like of an employee, for example.

Further, when grouping is performed by using attribute data of each employee, for example, the grouping unit 17 may classify employees having similar attribute data into a same group by using a general grouping method such as a K-MEANS clustering technique.

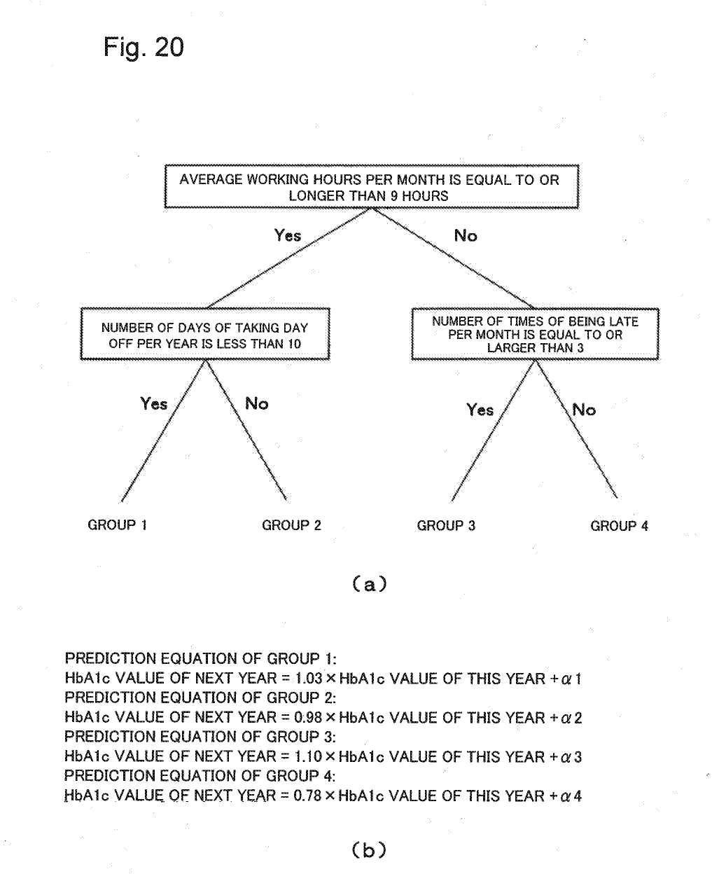

Further, as illustrated in FIG. 20(a), the grouping unit 17 may classify employees into groups, based on a predetermined condition expressed by a value of attribute data, or a condition designated by an advisor.

When employees are grouped, a prediction equation for calculating a predicted value of a target field for each group is provided (see FIG. 20(b)). Note that in FIG. 20(b), α1 to α4 denote segments of respective prediction equations.

In such a case, a predicting unit 16 may not only calculate a predicted value of a target field of an employee by using a prediction equation of a group to which the employee belongs, but also calculate a predicted value of a target field of the employee by using a prediction equation of another group. This allows an advisor to easily recognize a group to which the employee is likely to belong, from among groups in which a predicted value of a target field is included in a target range, based on a predicted value of a target field of each group and a condition for use in grouping. This is helpful when an item to be improved such as a work condition is pointed out. Note that the predicting unit 16 may perform processing of calculating a predicted value of a target field of each group, even when another grouping method is used.

Further, when grouping is performed by using medical examination data of each employee, the grouping unit 17 may classify employees having a similar content of medical examination data into a same group by using a general grouping method such as a K-MEANS clustering technique, for example. In addition, for example, as exemplified by the first modification example, when two types of medical examination data are present, employees having a similar magnitude of difference may be classified into a same group by obtaining a difference between first medical examination data and second medical examination data of each employee.

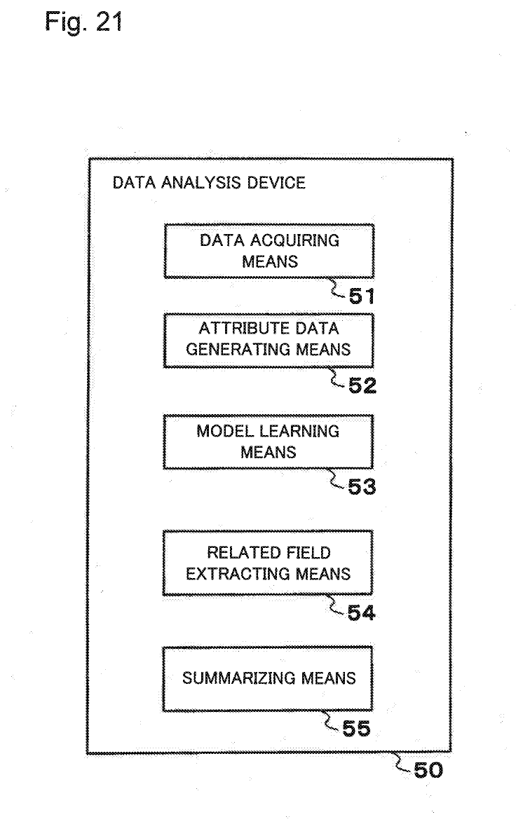

Next, a summary of the disclosed subject matter is described. FIG. 21 is a block diagram illustrating a summary of a data analysis device according to the disclosed subject matter. As illustrated in FIG. 21, a data analysis device 50 according to the disclosed subject matter includes a data acquiring means 51, an attribute data generating means 52, a model learning means 53, a related field extracting means 54, and a summarizing means 55.

The data acquiring means 51 (e.g. data input unit 11) acquires at least designation of a target field being a field from which relevance is extracted, from among fields included in health condition data being information relating to health conditions of an employee, the health condition data of two or more employees, and attendance data being information relating to a work condition.

The attribute data generating means 52 (e.g. attribute data generating unit 12) performs aggregation with respect to a predetermined field included in the attendance data of each employee by using a predetermined temporal resolution, a predetermined time range, and a predetermined aggregation method, and generates attribute data including each of aggregation results as an attribute field.

The model learning means 53 (e.g. model learning unit 13) learns a model, in which the target field is an object variable, and each of attribute fields included in attribute data is an explanatory variable, the model being represented by a polynomial, by using a content of the target field of the health condition data, and a content of the attribute data of the two or more employees.

The related field extracting means 54 (e.g. related field extracting unit 14) extracts an attribute field represented by a learned model and associated with the target field.

The summarizing means 55 (e.g. summarizing unit 15) summarizes and outputs attendance data of a designated employee, based on information on the extracted attribute field.

According to the aforementioned configuration, it is possible to obtain concrete field information associated with a designated field, without specifically designating an appropriate temporal resolution, an appropriate time range, and an appropriate aggregation method with respect to a field within attendance data.

Further, the model learning means may learn a coefficient of each of explanatory variables included in the polynomial, as a model parameter; and the related field extracting means may extract an attribute field associated with an explanatory variable having a value of the coefficient other than zero, as an attribute field associated with the target field.

Further, the attribute data generating means may perform aggregation with respect to one field of the attendance data by using a plurality of temporal resolutions, a plurality of time ranges, or a plurality of aggregation methods.

The data acquiring means may acquire designation of two or more target fields, and the model learning means may learn, regarding each of two or more designated target fields, a model, in which the target field is an object variable, and each of the attribute fields included in the attribute data is an explanatory variable, the model being represented by a polynomial, by using a content of the target field of health condition data, and a content of the attribute data of the two or more employees.

Further, the attendance data may include records for a first period before a first point of time being a predetermined point of time dating back from a predicted point of time being a predetermined future point of time by a predetermined second period, and records for a first period before a second point of time being a predetermined point of time dating back from a day when latest health condition data are acquired by the second period or longer. The attribute data generating means may perform aggregation with respect to a predetermined field included in second attendance data constituted by records for a first period before the second point of time for each of the employees by using a predetermined temporal resolution, a predetermined time range, and a predetermined aggregation method; and may generate second attribute data including each of aggregation results as an attribute field. The model learning means may learn a model, in which a target field of the latest health condition data is an object variable, and each of attribute fields included in the second attribute data is an explanatory variable, the model being represented by a polynomial, by using a content of a target field of the latest health condition data and a content of the second attribute data of two or more employees. The summarizing means may summarize first attendance data constituted by records for a first period before a first point of time of a designated employee, based on extracted attribute field information, and may output a summary result, as attendance data information associated with a target field at the predicted point of time.

Further, the data analysis device 50 may further include a predicting means (not illustrated, e.g., predicting unit 16) for predicting, based on the learned model, and first attribute data being attribute data to be generated by using first attendance data of a designated employee, a value of a target field at a predicted point of time of the employee.

Further, the data analysis device 50 may further include a grouping means (not illustrated, e.g., grouping unit 17) for grouping the employees, based on a predetermined condition, health condition data, attendance data, or attribute data. The model learning means may learn a model for each group of the employees by using a content of a target field of health condition data, and a content of attribute data of an employee belonging to the group.

Further, in the data analysis device 50, the attribute data may include an attribute field being a field included in health condition data, and in which an aggregation result with respect to a predetermined field other than a target field is registered. In such a case, the attribute data generating means may perform aggregation with respect to a predetermined field included in the attendance data, and a predetermined field being a field included in the health condition data and other than a target field for each of the employees by using a predetermined temporal resolution, a predetermined time range, and a predetermined aggregation method; and may generate attribute data including each of aggregation results as an attribute field. The summarizing means may summarize and output attendance data and the health condition data of the designated employee, based on extracted attribute field information.

As described above, the disclosed subject matter is described with reference to an example embodiment and examples. The disclosed subject matter, however, is not limited to the aforementioned example embodiment and examples. A configuration and details of the disclosed subject matter may be modified in various ways comprehensible to a person skilled in the art within the scope of the disclosed subject matter.

### INDUSTRIAL APPLICABILITY

The disclosed subject matter is not limited to providing field information associated with any medical examination result in attendance data for the purpose of health guidance, and is advantageously applicable to analyzing relevance between data including many fields and records, and any item.

This application claims the priority based on Japanese Patent Application No. 2015-142404 filed on Jul. 16, 2015, entire disclosure of which is hereby incorporated.

### REFERENCE SIGNS LIST

10 Data analysis device
11 Data input unit
12 Attribute data generating unit
13 Model learning unit
14 Related field extracting unit
15 Summarizing unit
16 Predicting unit
17 Grouping unit
50 Data analysis device
51 Data acquiring means
52 Attribute data generating means
53 Model learning means
54 Related field extracting means
55 Summarizing means
1001 CPU
1002 Memory
1003 Output device
1004 Input device
1005 Network interface

What is claimed is:

1. A data analysis device comprising:
data acquiring unit that acquires a at least designation of a target field being a field from which relevance is to be extracted, from among fields included in health condition data being information relating to a health condition of an employee, and the health condition data of two or more employees and attendance data being information relating to a work condition;
attribute data generating unit that performs aggregation with respect to a predetermined field included in the attendance data of each of the employees by using a predetermined temporal resolution, a time range, and an aggregation method, and generates attribute data including each of aggregation results as an attribute field;
model learning unit that learns a model, in which the target field is an object variable, and each of attribute fields included in the attribute data is an explanatory variable, the model being represented by a polynomial, by using a content of the target field of the health condition data, and a content of the attribute data, of the two or more employees;
related field extracting unit that extracts an attribute field represented by a learned model and associated with the target field; and
summarizing unit that summarizes and outputs attendance data of a designated employee, based on information on the extracted attribute field.

2. The data analysis device according to claim 1,
wherein the model learning unit learns a coefficient of each of explanatory variables included in the polynomial, as a model parameter, and
wherein the related field extracting unit extracts an attribute field associated with an explanatory variable having a value of the coefficient other than zero, as an attribute field associated with the target field.

3. The data analysis device according to claim 1,
wherein the attribute data generating unit performs aggregation with respect to one field of the attendance data by using a plurality of temporal resolutions, a plurality of time ranges, or a plurality of aggregation methods.

4. The data analysis device according to claim 1,
wherein the data acquiring unit acquires designation of two or more target fields, and
wherein the model learning unit learns, regarding each of two or more designated target fields, a model, in which the target field is an object variable, and each of the attribute fields included in the attribute data is an explanatory variable, the model being represented by a polynomial, by using a content of the target field of health condition data, and a content of the attribute data, of the two or more employees.

5. The data analysis device according to claim 1,
wherein the attendance data include records for a first period before a first point of time being a predetermined point of time dating back from a predicted point of time being a predetermined future point of time by a predetermined second period, and records for a first period before a second point of time being a predetermined point of time dating back from a day when latest health condition data are acquired by the second period or longer,
wherein the attribute data generating unit performs aggregation with respect to a predetermined field included in second attendance data constituted by records for a first period before the second point of time for each of the employees by using a predetermined temporal resolution, a time range, and an aggregation method, and generates second attribute data including each of aggregation results as an attribute field,
wherein the model learning unit learns a model, in which a target field of the latest health condition data is an object variable, and each of attribute fields included in the second attribute data is an explanatory variable, the model being represented by a polynomial, by using a content of a target field of the latest health condition data, and a content of the second attribute data, of two or more employees, and
wherein the summarizing unit summarizes first attendance data constituted by records for a first period before a first point of time of a designated employee, based on extracted attribute field information, and outputs a summary result, as attendance data information associated with a target field at the predicted point of time.

6. The data analysis device according to claim 5, further comprising
predicting unit that predicts, based on the learned model and first attribute data being attribute data generated by using first attendance data of a designated employee, a value of a target field at a predicted point of time of the employee.

7. The data analysis device according to claim 1, further comprising
grouping unit that generates the employees, based on a predetermined condition, health condition data, attendance data, or attribute data,
wherein the model learning unit learns a model for each group of the employees by using a content of a target field of health condition data, and a content of attribute data, of an employee belonging to the group.

8. The data analysis device according to claim 1,
wherein the attribute data include an attribute field being a field included in health condition data, in which an aggregation result with respect to a predetermined field other than a target field is registered,
wherein the attribute data generating unit performs aggregation with respect to a predetermined field included in the attendance data, and a predetermined field being a field included in the health condition data and other than a target field for each of the employees by using a predetermined temporal resolution, a time range, and an aggregation method, and generates attribute data including each of aggregation results as an attribute field, and
wherein the summarizing unit summarizes and outputs attendance data and the health condition data of the designated employee, based on extracted attribute field information.

9. A data analysis method comprising:
causing an information processing device to acquire at least designation of a target field being a field from which relevance is to be extracted, from among fields included in health condition data being information relating to a health condition of an employee, and the health condition data of two or more employees and attendance data being information relating to a work condition;
causing the information processing device to perform aggregation with respect to a predetermined field included in the attendance data of each of the employees by using a predetermined temporal resolution, a time range, and an aggregation method, and to generate attribute data including each of aggregation results as an attribute field;
causing the information processing device to learn a model, in which the target field is an object variable, and each of attribute fields included in the attribute data is an explanatory variable, the model being represented by a polynomial, by using a content of the target field of the health condition data, and a content of the attribute data, of the two or more employees;
causing the information processing device to extract an attribute field represented by a learned model and associated with the target field; and
causing the information processing device to summarize and output attendance data of a designated employee, based on information on the extracted attribute field.

10. A non-transitory computer readable storage medium storing a data analysis program which causes a computer to execute:
processing of acquiring at least designation of a target field being a field from which relevance is to be extracted, from among fields included in health condition data being information relating to a health condition of an employee, and the health condition data of two or more employees and attendance data being information relating to a work condition;
processing of performing aggregation with respect to a predetermined field included in the attendance data of each of the employees by using a predetermined temporal resolution, a time range, and an aggregation method, and generating attribute data including each of aggregation results as an attribute field;
processing of learning a model, in which the target field is an object variable, and each of attribute fields included in the attribute data is an explanatory variable, the model being represented by a polynomial, by using a content of the target field of the health condition data, and a content of the attribute data, of the two or more employees;
processing of extracting an attribute field represented by a learned model and associated with the target field; and
processing of summarizing and outputting attendance data of a designated employee, based on information on the extracted attribute field.
